(19) United States
(12) Patent Application Publication
(10) Pub. No.: US 2025/0073138 A1
(43) Pub. Date: Mar. 6, 2025

(54) Lipid-based nanoparticles for enhancing percutaneous absorption and manufacturing method thereof

(71) Applicants: COSMECCA KOREA CO., LTD., Chungcheongbuk-do (KR); Research & Business Foundation SUNGKYUNKWAN UNIVERSITY, Suwon-si (KR)

(72) Inventors: Ji Soo Ryu, Anyang-si (KR); Jae Yong Seo, Cheongju-si (KR); Hang Eui Cho, Yongin-si (KR); Hyun Dae Cho, Cheongju-si (KR); Jin Woong Kim, Seongnam-si (KR); Minha Jeon, Suwon-si (KR); Seulgi Kim, Suwon-si (KR); Se Eun Kim, Seoul (KR)

(21) Appl. No.: 18/778,212

(22) Filed: Jul. 19, 2024

(30) Foreign Application Priority Data
Jul. 21, 2023 (KR) 10-2023-0095306

(51) Int. Cl. A61K 8/14; B82Y 5/00

(52) U.S. Cl. CPC A61K 8/14; A61K 2800/413; A61K 2800/612; A61K 2800/805; B82Y 5/00

(57) ABSTRACT

The present invention relates to a lipid-based nanoparticle composition for enhancing transdermal absorption and a method of preparing the same. The present invention provides a carrier for enhancing transdermal absorption that enhances or promotes chemical bonding with skin tissue by modifying the surface of lipid nanoparticles with a polyphenol. This may demonstrate the synergistic effect of active and passive transdermal delivery systems.

### BACKGROUND

### 1. Field of the Invention

The present invention relates to a lipid-based nanoparticle composition for enhancing transdermal absorption and a method of preparing the same.

### 2. Discussion of Related Art

The skin is composed of three layers: the epidermis, dermis, and subcutaneous fat. Among these, the stratum corneum, located in the outermost layer of the epidermis, prevents foreign materials from entering from the outside and moisture from evaporating. It acts as a protective skin barrier layer, but has lower material permeability compare to general biological membranes. Therefore, in order to effectively deliver active ingredients to skin cells, the drug must pass through the stratum corneum and be delivered to the layer of active cells in the epidermis.

A transdermal delivery system utilizes a drug delivery method that delivers active ingredients needed by the body through the skin using various physical and chemical technologies. This represents a newly developed drug delivery method following traditional approaches such as oral administration and intravenous injection. In particular, it can effectively alleviate side effects that may occur with oral administration, and related research is being actively conducted. Currently, research on transdermal delivery systems has led to the development of physical methods, such as microneedles and electrophoresis, which require patches or mechanical devices for delivery. As for chemical methods, the development of passive transdermal delivery systems based on nanocarriers is in progress, using polymer micelles and nanoemulsions to reduce particle size or enhance stability. However, these nanocarriers have the disadvantage of being difficult to pass through the stratum comeum, resulting in low delivery efficiency.

To address this limitation, transdermal delivery systems have been developed that mix various additives to achieve a strong chemical enhancement effect. Nanocarrier-based transdermal delivery systems with improved transdermal efficiency include ethosomes, which are ethanol-containing vesicles, and transfersomes, which are vesicles containing a single-chain surfactant (edge activator). However, these systems still face challenges in penetrating the skin. Therefore, it is necessary to develop a drug carrier that compensates for these drawbacks, promotes the absorption of active ingredients without irritating the skin, and maximizes the overall effect.

### RELATED ART DOCUMENT

### Patent Document

(Patent Document 0001) KR 10-2011-0114202 A

### SUMMARY OF THE INVENTION

The present invention is aimed at providing a nanoparticle transdermal delivery system that can effectively deliver biologically active ingredients, such as whitening, wrinkle improvement, and antioxidation agents, to the active cell layer beneath the stratum corneum.

The inventors completed this invention by confirming that biologically active ingredients incorporated into lipid nanoparticles are effectively delivered to the active cell layer beneath the stratum corneum. This is achieved by the strong binding of polyphenols, attached to the surface of the lipid nanoparticles, to the extracellular matrix and cell surface proteins through hydrogen bonding.

The present invention provides lipid nanoparticles surface-modified with a polyphenol.

In this specification, “polyphenol” has the property of being able to bind strongly to extracellular matrix and cell surface proteins through hydrogen bonding. Specifically, in the present invention, polyphenols may be one or more selected from the group consisting of hydroxybenzoic acid-based compounds, hydroxycinnamic acid-based compounds, flavonoid-based compounds, lignan-based compounds, stilbene-based compounds, caffeic acid, chlorogenic acid, anthocyan, pyrogallol, ellagic acid, gallic acid, ellagic acid, catechin, hydrolyzable tannin, condensed tannin, and theaflavin-3-gallate. More specifically, the polyphenols may be one or more selected from the group consisting of tannic acid, flavonoids, ellagitannins, catechins, quercetin, isoflavones, anthocyanins, proanthocyanins, and proanthocyanidins, but are not limited thereto.

In this specification, “lipid nanoparticle” refers to a nano-sized particle composed of a lipid material, meaning a particle coated with lipid or a lipid-related material, and usually contains a lipid, which is a lipid compound, as a main component.

The main components of lipid nanoparticle may comprise lipid materials such as phospholipids, neutral lipids, and a single lipid layer, and specifically, the lipid nanoparticles of the present invention may be produced from phospholipids.

In this specification, “phospholipid” is a component of biological membranes, and is an amphiphilic material that exhibits both hydrophilic and hydrophobic properties as it contains a phosphate group, and may be mainly used in the production of lipid nanoparticles that deliver poorly soluble materials. The phospholipid may be a glycerophospholipid or a sphingophospholipid, and may specifically be a glycerophospholipid.

As used herein, “glycerophospholipid” may be one or more of phosphatidic acid (PA), phosphatidylethanolamine (PE), phosphatidylcholine (PC), phosphatidylserine (PS), phosphatidylinositol (PI), phosphatidylinositol phosphate (PIP), phosphatidylinositol bisphosphate (PIP2), and phosphatidylinositol trisphosphate (PIP3), and specifically phosphatidylcholine (PC), but is not limited thereto.

Some glycerophospholipids constituting the lipid nanoparticle of the present invention may be thiol (—SH)-substituted glycerophospholipids.

The thiol-substituted glycerophospholipids may be selected from the group consisting of dipalmitoyl phosphatidyl thioethanol, distearoyl phosphatidyl thioethanol, dioleoyl phosphatidyl thioethanol, dimyristoyl phosphatidyl thioethanol, and a combination thereof.

The lipid nanoparticle of the present invention may comprise glycerophospholipids and thiol-substituted glycerophospholipids in a weight ratio of 5 to 10:1. When the lipid nanoparticles are prepared outside the above weight ratio, they may not be formed or surface modification with a polyphenol may not occur.

The lipid nanoparticles of the present invention may be surface-modified with a polyphenol, specifically tannic acid, and may be formed by binding the maleimide group of the tannic acid-maleimide complex represented by the following Chemical Formula 1 to the thiol (—SH) group of a phospholipid substituted with a thiol group:

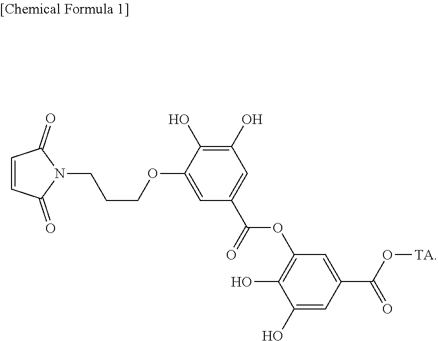

The size of the lipid nanoparticle surface-modified with a polyphenol, specifically tannic acid, of the present invention may be 50 to 200 nm, preferably 100 to 150 nm.

The lipid nanoparticles of the present invention may be prepared through a film hydration method. Here, the ‘film hydration method’ refers to dissolving lipid components in an organic solvent, forming a lipid film through solvent evaporation, hydrating the film, and applying physical force to produce nano-sized particles.

Therefore, the present invention can produce lipid nanoparticles surface-modified with a polyphenol, specifically tannic acid, through the following process.
(1) producing glycerophospholipid-based lipid nanoparticles using a film hydration method;
(2) preparing a polyphenol-maleimide complex; and
(3) dispersing the polyphenol-maleimide complex on the surface of the glycerophospholipid-based lipid nanoparticles of step (1).

The step (1) can comprise dissolving phosphatidylcholine, which is a glycerophospholipid, and dipalmitoyl phosphatidyl thioethanol, which is a glycerophospholipid substituted with thiol, in a solvent and then removing the solvent to prepare a lipid film; and inducing self-aggregation between the lipid components while peeling off the lipid film using ultrasound.

In step (1), glycerophospholipids and thiol-substituted glycerophospholipids may be used in a weight ratio of 5 to 10:1.

In order to increase the transdermal absorption efficiency of the lipid nanoparticles produced in step (1), a polyphenol whose terminal is modified with a maleimide group may be synthesized as in step (2) to introduce tannic acid, which is a polyphenol material, to the surface. This proceeds through two processes. The first process produces N-3-bromopropyl maleimide through the organic reaction of 3-bromopropylamine hydrobromide and triethanolamine with maleic anhydride. Next, a polyphenol-maleimide complex is synthesized by modifying the end of the polyphenol with a maleimide group using the produced N-3-bromopropyl maleimide, polyphenol, and potassium carbonate.

In step (3), the polyphenol may be attached to the surface of the lipid nanoparticles produced in step (1) through a reaction between thiol group and maleimide.

The lipid nanoparticles prepared by the above-described film hydration method may have a size of 30 to 200 nm, preferably 100 to 150 nm, depending on the amount of the polyphenol-maleimide complex introduced.

The lipid nanoparticles of the present invention may be produced by incorporating various materials that exhibit physiological activity in the fields of cosmetics and pharmaceuticals. Therefore, lipid nanoparticles carrying such physiologically active materials can be used as carriers, specifically as transdermal absorption carriers. As the lipid nanoparticles of the present invention are modified with a polyphenol, specifically tannic acid, they adhere better to the extracellular matrix, thereby improving the transdermal absorption performance of both the lipid nanoparticles and the biologically active materials incorporated within them.

### BRIEF DESCRIPTION OF THE DRAWINGS

The above and other objects, features and advantages of the present invention will become more apparent to those of ordinary skill in the art by describing exemplary embodiments thereof in detail with reference to the accompanying drawings, in which:

FIG. 1 is a schematic diagram of a method of producing nanoparticles modified with tannic acid:

FIGS. 2A-2C show (FIG. 2A) the synthesis method of tannic acid-maleimide and the 1H NMR results of (FIG. 2B) N-3-bromopropyl maleimide and (FIG. 2C) TAMA:

FIGS. 3A-3B show (FIG. 3A) the reaction of TAMA and 16:0 Ptd thioethanol and (FIG. 3B) the ¹³C NMR results of TAMA and TANV;

FIG. 4 shows a TEM image of TANV:

FIGS. 5A-5B show the results of comparing (FIG. 5A) the size and surface potential changes of TANV by introduction concentration of TAMA and (FIG. 5B) the size distribution of TANV with nanoparticles and TAMA attached at a concentration of 50 μM:

FIG. 6 shows the results of evaluating the cytotoxicity of TA, TAMA, and TANV by the introduction concentration of tannic acid through live/dead assay:

FIGS. 7A-7B show the results of comparing (FIG. 7A) uv-vis results of TANV and a mixture of TANV and collagen and (FIG. 7B) the difference in absorbance of TANV and a mixture of TANV and collagen at 600 nm:

FIGS. 8A-8C show (FIG. 8A) an uptake image of TANV into HaCaT cells, (FIG. 8B) the results of uptake fluorescence values of HaCaT cells when TANV was treated by the introduction concentration of TAMA, and (FIG. 8C) the results of uptake fluorescence values of nanoparticles and TANV in HaCaT cells over time; and

FIGS. 9A-9C show (FIG. 9A) the confocal microscopy tomographic image of porcine skin treated with NR-TANV and (FIG. 9B) calculated fluorescence amount by depth, and (FIG. 9C) the results of calculating the fluorescence value for each introduction concentration of TAMA.

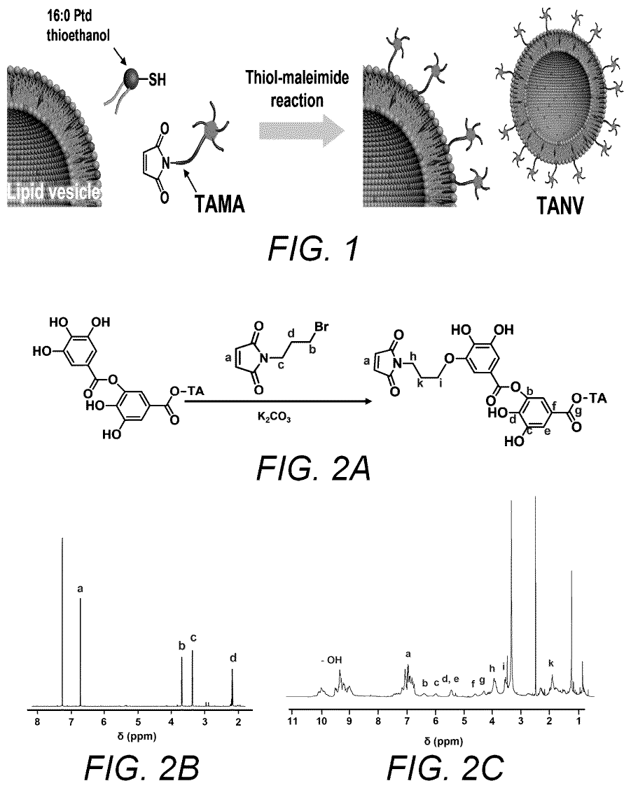

### DETAILED DESCRIPTION OF EXEMPLARY EMBODIMENTS

Hereinafter, a detailed description of preferred embodiments of the present invention will be described with reference to the attached drawings. However, the present invention is not limited to the experimental examples described herein, and may be embodied in other forms. Rather, the embodiments introduced herein are provided so that the disclosure will be thorough and complete, and so that the spirit of the invention can be sufficiently conveyed to those skilled in the art. In describing the present invention below, if a detailed description of a related known function or configuration is judged to unnecessarily obscure the subject matter of the present invention, the detailed description may be omitted.

### <Preparation Example 1> Synthesis of Tannic Acid-Maleimide (TAMA)

Tannic acid-maleimide (TAMA) was synthesized by modifying the terminal of tannic acid with a maleimide group. First, to synthesize the intermediate N-3-bromopropylmaleimide, 3-bromopropylamine hydrobromide, triethanolamine, and maleic anhydride were mixed with dichloromethane and stirred for 24 h. Thereafter, residual impurities were removed through a separatory funnel, an organic layer was obtained, and the solvent was removed through a rotary evaporator. Thereafter, sodium acetate anhydride and acetic anhydride were added to the generated material and stirred under reflux for 24 h. Thereafter, it was neutralized with sodium bicarbonate and the organic layer was extracted with dichloromethane. The solvent of the generated organic layer was removed through the rotary evaporator, and purified N-3-bromopropylmaleimide was obtained through column chromatography. In order to attach tannic acid to the prepared intermediate, N-3-bromopropylmaleimide, tannic acid, and potassium carbonate were stirred together in dimethylformamide at 60° C. for 24 h. Thereafter, dialysis was performed for 4 days to replace the solvent with double distilled water, and the generated aqueous solution was lyophilized to obtain tannic acid-maleimide (maleimide-functionalized TA, TAMA) as a yellow-brown solid.

### <Preparation Example 2> Preparation of Tannic Acid-Modified Nanoparticles (TANV)

Biolipid-based nanoparticles were produced through a film hydration method. First, phosphatidylcholine and 16:0 ptd thioethanol were quantified to be 2 mg/ml and 0.3 mg/ml, respectively, relative to the final solution. Thereafter, the corresponding lipids were dissolved in chloroform and then the solution was placed in a round bottom flask. Thereafter, chloroform was evaporated through the rotary evaporator, and a film composed of lipids was obtained. Thereafter, a PBS buffer solution was added and treated with a probe tip ultrasonicator under 50% intensity conditions for 5 min, and the film was hydrated to produce lipid-based nanoparticles. TAMA prepared in Preparation Example 1 was added to the nanoparticle dispersion and stirred for 24 h to prepare nanoparticles surface-modified with tannic acid (TANV). At this time, 0, 25, 50, 75, and 100 μM of TAMA was introduced into the nanoparticles, and nanoparticles surface-modified with tannic acid were prepared at different concentrations shown in Table 1 below.

TABLE 1

| | Polyphenolic material | Concentration (μM) |
|---|---|---|
| Comparative Example 1 | No additives | 0 |
| Example 1 | TAMA | 25 |
| Example 2 | | 50 |
| Example 3 | | 75 |
| Example 4 | | 100 |

### Experimental Example 1

To confirm that N-3-bromopropylmaleimide, an intermediate of tannic acid-maleimide (TAMA), and TAMA in Preparation Example 1 were synthesized, structural analysis was performed using hydrogen nuclear magnetic resonance (1H NMR) spectroscopy, and this is shown in FIGS. 2A-2C.

### Experimental Example 2

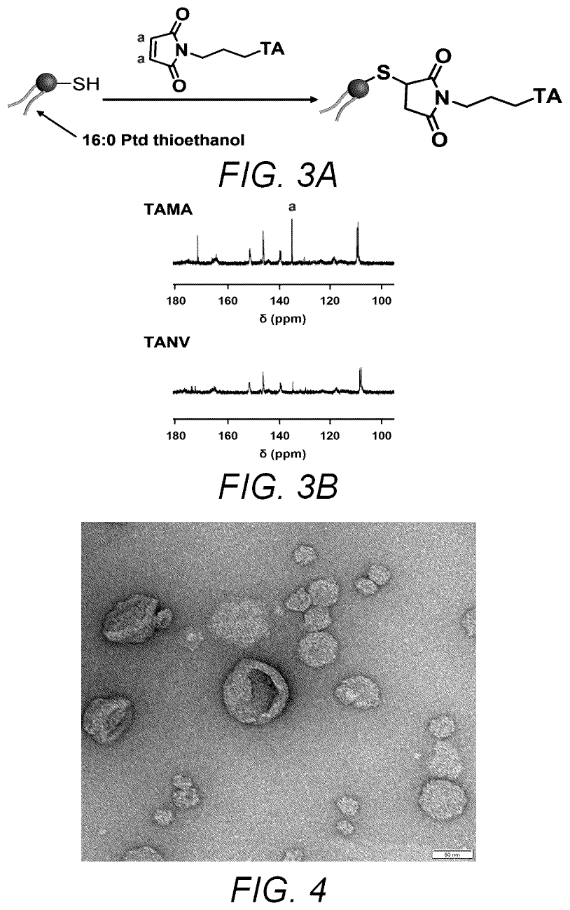

To confirm that the maleimide group of TAMA was reacted with 16:0 ptd thioethanol of the nanoparticle, structural analysis was performed using carbon nuclear magnetic resonance (¹³C NMR) spectroscopy. As a result, it was confirmed that the maleimide group peak (position: 134.95 ppm) of TAMA in TANV, a tannic acid-modified nanoparticle, was reduced, which is shown in FIGS. 3A-3B.

### Experimental Example 3

The shape of the tannic acid-modified nanoparticles was confirmed through transmission electron microscope (TEM) images, which is shown in FIG. 4.

### Experimental Example 4

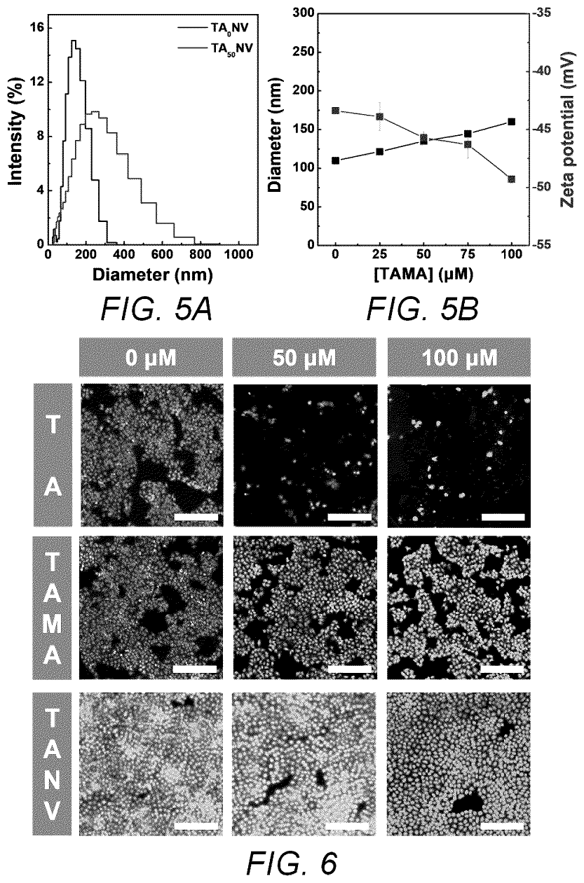

The size and surface potential that change as TAMA is modified into nanoparticles were confirmed using a zeta sizer (DTS 1070, Malvern, UK), and the size distribution of nanoparticles without TAMA attached (Comparative Example 1) and TANV with 50 μM of TAMA attached to the nanoparticles (Example 2) was confirmed. Depending on the introduction concentration of TAMA, there was a size change of about 100 nm to 150 nm, and a surface potential change of about-43 mV to −50 mV. This is shown in FIG. 5A and FIG. 5B.

### Experimental Example 5

Live/dead fluorescence staining was used to confirm the safety of TAMA and TANV with TAMA modified on the surface. First, HaCaT, a human keratinocyte cell line, was treated with tannic acid, TAMA, and TAMA-introduced nanoparticles (TANV) at different concentrations for 24 h. Thereafter, calcein-acetoxymethyl ester (calcein-AM), which stains viable cells, and ethidium homodimer, which stains dead cells, were treated simultaneously, and then washed twice with the PBS buffer solution to obtain images of viable and dead cells through fluorescence microscopy.

It was found that when tannic acid was treated as is without modification, cytotoxicity and death occurred significantly due to the induction of reactive oxygen species (ROS). However, when TAMA and TANV samples were treated, safety was demonstrated by showing a slight level of cytotoxicity, which is shown in FIG. 6.

### Experimental Example 6

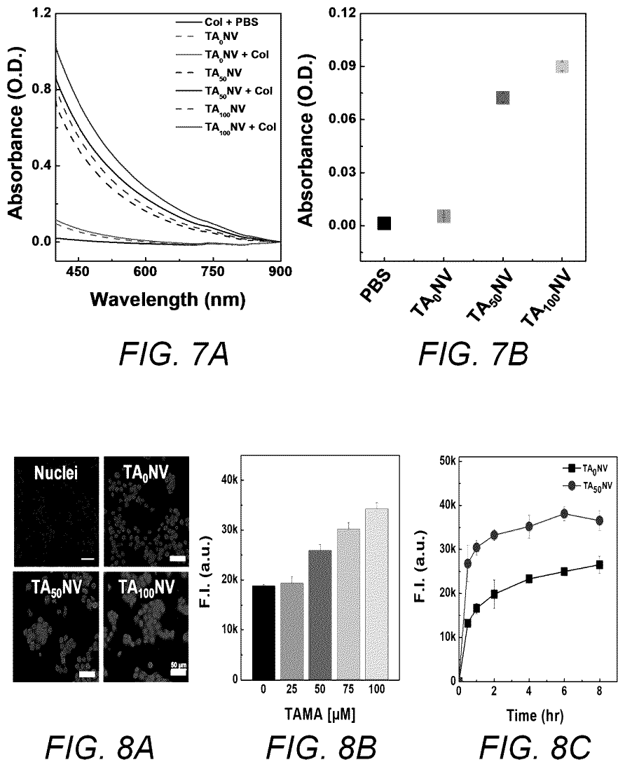

To determine whether the extracellular matrix adhesive performance of TANV increases depending on the amount of TAMA introduced, collagen and TANV dispersions were stirred and turbidity was measured. The turbidity was measured using absorbance UV-Vis spectroscopy. As a result, it was found that as the amount of TAMA introduced increased, the binding force with collagen increased and agglomeration occurred, thereby increasing the turbidity of the solution. Through this, it was confirmed that the nanoparticles had effective adhesive performance with the extracellular matrix by introducing TAMA, and this is shown in FIGS. 7A-7B.

### Experimental Example 7

To confirm that the uptake of skin cells increases depending on the amount of TAMA introduced in TANV, TANV (TR-TANV) containing Texas Red was prepared. At this time, the amount of TAMA introduced was adjusted to 0, 25, 50, 75, and 100 μM, which is shown in Table 2 below.

TABLE 2

| | Polyphenolic | Concentration | Fluorescent |
|---|---|---|---|
| | material | (μM) | material |
| Comparative | No additives | 0 | Texas Red |
| Example 1 | | | |
| Example 1 | TAMA | 25 | Texas Red |
| Example 2 | | 50 | Texas Red |
| Example 3 | | 75 | Texas Red |
| Example 4 | | 100 | Texas Red |

Thereafter, HaCaT cells were cultured in a 12-well plate, and the prepared TR-TANV was diluted in medium and treated for 6 h. Thereafter, cells were fixed with a formalin solution, treated with DAPI fluorescent dye solution for 5 minutes, then washed twice with the PBS buffer solution, and TR-TANV absorbed into cell nuclei and cells were observed through a fluorescence microscope.

HaCaT cells were cultured in a 96-well plate, and Comparative Example 1 and Examples 1 to 4 were treated for 6 h, and then washed twice with the PBS buffer solution. Thereafter, the fluorescence intensity was quantified at Ex/Em=535/595 nm using a microplate reader.

After culturing HaCaT cells in a 96-well plate, Comparative Example 1 and Example 2 were treated over time from 0 to 8 h, and then washed twice with the PBS buffer solution. Thereafter, the fluorescence intensity was quantified at Ex/Em=535/595 nm using a microplate reader.

Through this, depending on the amount of TAMA introduced, it was found that when a 50 μM concentration of TAMA was introduced into the nanoparticles (Example 2), the uptake of TANV in keratinocytes increased by about 38%, and when a 100 μM concentration of TAMA was introduced into the nanoparticles (Example 4), by about 82%, which is shown in FIG. 8A and FIG. 8B. In addition, the difference in TANV absorption over time between particles with TAMA introduced (Example 2) and particles without TAMA (Comparative Example 1) was confirmed. In other words, it was confirmed that the absorption amount of particles with TAMA introduced increased relatively significantly over time, as shown in FIG. 8C.

### Experimental Example 8

To confirm that the transdermal absorption performance of TANV increases depending on the amount of TAMA introduced, TANV (NR-TANV) containing Nile Red was prepared. At this time, the amount of TAMA introduced was adjusted to 0, 25, 50, 75, and 100 μM, which is shown in Table 3 below.

TABLE 3

| | Polyphenolic | Concentration | Fluorescent |
|---|---|---|---|
| | material | (μM) | material |
| Comparative | No additives | 0 | Nile Red |
| Example 1 | | | |
| Example 1 | TAMA | 25 | Nile Red |
| Example 2 | | 50 | Nile Red |
| Example 3 | | 100 | Nile Red |

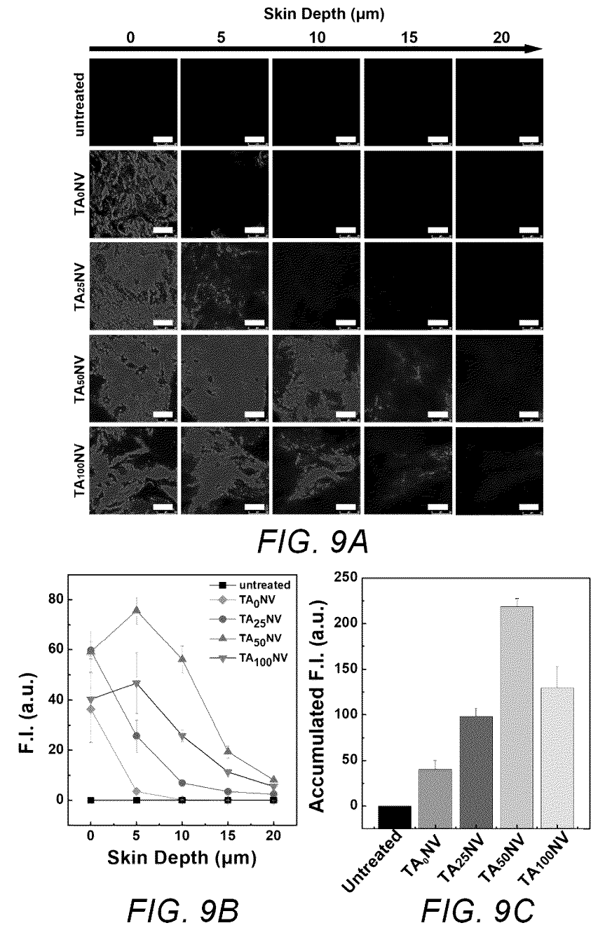

To confirm the transdermal absorption performance of TANV, the surface of porcine skin measuring 1*1 cm² was treated with TANV (NR-TANV) containing Nile Red for 24 h, and then tomography was performed by skin depth using a confocal microscope, which is shown in FIG. 9A. In addition, as a result of checking the fluorescence amount by depth and the fluorescence value by introduction concentration of TAMA, it was confirmed that when 50 μM concentration of TAMA was introduced into the nanoparticles (Example 2), the transdermal absorption depth increased by about 15 μm compared to the particles without TAMA (Comparative Example 1). However, when 50 UM or more of TAMA was introduced, the absorption depth did not increase, confirming that the 50 μM concentration was optimal, which is shown in FIG. 9B and FIG. 9C. Through this, the excellent transdermal absorption performance of nanoparticles modified with tannic acid was confirmed.

### Advantageous Effects

The present invention provides a carrier for enhancing transdermal absorption that enhances or promotes chemical bonding with skin tissue by modifying the surface of lipid nanoparticles with a polyphenol. This may demonstrate the synergistic effect of active and passive transdermal delivery systems.

What is claimed is:

1. A lipid nanoparticle surface-modified with a polyphenol.

2. The lipid nanoparticle of claim 1, wherein the lipid nanoparticle comprises glycerophospholipids.

3. The lipid nanoparticle of claim 2, wherein some of the glycerophospholipids are thiol (—SH)-substituted glycerophospholipids.

4. The lipid nanoparticle of claim 3, wherein the thiol-substituted glycerophospholipid is selected from the group consisting of dipalmitoyl phosphatidyl thioethanol, distearoyl phosphatidyl thioethanol, dioleoyl phosphatidyl thioethanol, dimyristoyl phosphatidyl thioethanol, and a combination thereof.

5. The lipid nanoparticle of claim 3, wherein the lipid nanoparticle comprises glycerophospholipids and thiol-substituted glycerophospholipids in a weight ratio of 5 to 10:1.

6. The lipid nanoparticles of claim 1, wherein the polyphenol is one or more selected from the group consisting of tannic acid, flavonoid, ellagitannin, catechin, quercetin, isoflavone, anthocyanin, and proanthocyanin.

7. The lipid nanoparticle of claim 2, wherein the surface modification is formed by combining the polyphenol with the thiol group of the thiol-substituted glycerophospholipids.

8. The lipid nanoparticle of claim 2, wherein the surface modification is formed by combining the maleimide group of the following Chemical Formula 1 with the thiol group of the thiol-substituted glycerophospholipids:

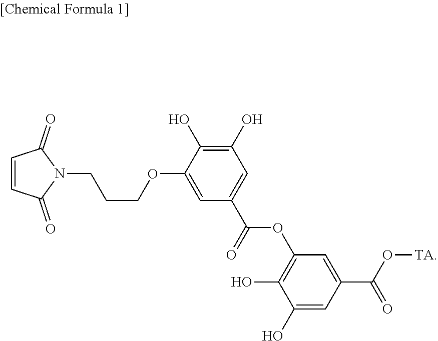

9. The lipid nanoparticle of claim 1, wherein the lipid nanoparticle has a size of 50 to 200 nm.

10. A carrier comprising:
the lipid nanoparticles of claim 1, and
a biologically active ingredient contained in the lipid nanoparticles.

11. The carrier according to claim 10, wherein the carrier increases the transdermal absorption rate of the biologically active ingredient.

12. A cosmetic composition for enhancing transdermal absorption comprising the lipid nanoparticles of claim 1.

13. The cosmetic composition of claim 12, wherein the lipid nanoparticles comprise a biologically active ingredient.

14. A method of producing lipid nanoparticles surface-modified with a polyphenol, comprising:
(1) producing glycerophospholipid-based lipid nanoparticles using a film hydration method;
(2) preparing a polyphenol-maleimide complex; and
(3) dispersing the polyphenol-maleimide complex on the surface of the glycerophospholipid-based lipid nanoparticles of step (1).

15. The method of claim 14, wherein, in step (1), the glycerophospholipid-based lipid nanoparticles are prepared by mixing glycerophospholipids and thiol-substituted glycerophospholipids in a weight ratio of 5 to 10:1.

16. The method of claim 14, wherein the polyphenol is one or more of tannic acid, flavonoid, ellagitannin, catechin, quercetin, isoflavone, anthocyanin, and proanthocyanin.
